(19) United States
(12) Patent Application Publication
(10) Pub. No.: US 2026/0355739 A1
(43) Pub. Date: Nov. 12, 2026

(54) DEVICES AND METHODS FOR QUANTITATIVE MEASUREMENT OF ONE OR MORE ANALYTES OVER A RANGE OF CONCENTRATIONS

(71) Applicant: University of Washington, Seattle, WA (US)

(72) Inventors: Paul YAGER, Seattle, WA (US); Sujatha KUMAR, Seattle, WA (US); Steven BENNETT, Seattle, WA (US); Erin K. HEINIGER, Seattle, WA (US); Kevin JIANG, Seattle, WA (US); Hugh ALESSI, Seattle, WA (US); Raghd Aidaros MHAVAL, Seattle, WA (US)

(21) Appl. No.: 19/147,095

(22) PCT Filed: Jan. 31, 2024
(86) PCT No.: PCT/US2024/013727

§ 371 Date: Jul. 10, 2025

Related U.S. Application Data
(60) Provisional application No. 63/482,823, filed on Feb. 2, 2023.
(60) Provisional application No. 63/545,706, filed on Oct. 25, 2023.

(51) Int. Cl. B01L 3/00

(52) U.S. Cl. CPC B01L 3/502707; B01L 3/502715; B01L 2300/0636

(57) ABSTRACT

Devices, systems, and methods for detecting an analyte in a sample, such as through measuring an analyte concentration in a sample, such as a fluid sample are described. In an embodiment, the devices comprise a fluidic pathway comprising a pathway input and a pathway outlet, wherein the fluidic pathway is configured to flow the fluid sample from the pathway input to the pathway output; a detection zone in fluidic communication with the pathway outlet; one or more reagent pads in fluidic communication with the fluidic pathway; a sample inlet port in fluidic communication with the fluidic pathway between the one or more reagent pads and the pathway outlet; and a plurality of detection spots disposed in the detection zone, wherein each detection spot of the plurality of detection spots comprises a density of capture molecules configured to selectively bind to the analyte.

### CROSS-REFERENCES TO RELATED APPLICATIONS

This application claims the benefit of U.S. Provisional Patent Application No. 63/482823, filed Feb. 2, 2023, and U.S. Provisional Patent Application No. 63/545706, filed Oct. 25, 2023; the contents of which are hereby incorporated by reference in their entireties for all purposes.

### BACKGROUND

Home or point-of-care (POC) detection of protein biomarkers that are present in biological samples over a wide range of concentrations presents problems for many conventional POC detection devices. This is a challenging problem for a single device, particularly one intended to be used in a home and cost very little.

Currently there are no known methods of measuring concentrations of target biomolecules (antigens (Ags), antibodies (Abs), proteins, lipids, etc.) over a wide range with only a single capture Ab species in a single capture region that do not employ serial dilutions of the sample.

### SUMMARY

To address these and related challenges, the present disclosure provides systems, devices, and methods for detection of analytes, such as protein analytes. In an embodiment, the present disclosure provides a device configured to capture and cause to be measurable concentrations of protein-sized biomarkers present over many orders of magnitude in concentration in a sample. In an embodiment, the device is coupled, configured to be coupled, or includes an imaging system, such as a cell phone camera, configured to generate image stacks collected over a period of time (~30 seconds, such as after a reaction time of approximately 30 minutes) for analysis to determine target analyte concentrations.

In an embodiment, the device is configured to perform sequential delivery microfluidics to convey multiple types of detection chemistry onto a common imageable detection zone. In an embodiment, the device is configured to sequentially deliver low-sensitivity detection technologies like AuNP with high-sensitivity detection chemistries, such as enzymatic amplification, to allow a single image to measure concentration of analytes over a wide range. In an embodiment, the device includes patterning of multiple collection spots in such a way as to avoid or reduce shielding of some detection spots from ones upstream. In an embodiment, the device includes detection spots created by depositing varied concentrations of spotting solutions (and, hence, varied local densities of capture molecules) in the final dried capture spots. In an embodiment, the device includes an imaging system configured to obtain or generate multiple timed images of chemically amplified spots to allow measurement of initial rate kinetics to provide better and faster estimates of analyte concentrations than would be possible from taking only end-point measurements.

Accordingly, in an aspect, the present disclosure provides an assay device for detection of an analyte in a fluid sample. In an embodiment, the assay device comprises a fluidic pathway comprising a pathway input and a pathway outlet, wherein the fluidic pathway is configured to flow the fluid sample from the pathway input to the pathway output; a detection zone in fluidic communication with the pathway outlet; one or more reagent pads in fluidic communication with the fluidic pathway; a sample inlet port in fluidic communication with the fluidic pathway between the one or more reagent pads and the pathway outlet; and capture molecules configured to selectively bind to the analyte and disposed in the detection zone, wherein the capture molecules comprise capture molecules comprising different binding affinities for the analyte or wherein the capture molecules are disposed in the detection zone defining detection regions comprising different densities of capture molecules.

In another aspect, the present disclosure provides a diagnostic system. In an embodiment, the diagnostic system comprises an assay device according to any embodiment of the present disclosure; a detection device; and a controller operatively coupled to the detection device. In an embodiment, the diagnostic system comprises an assay device comprising a fluidic pathway comprising a pathway input and a pathway outlet, wherein the fluidic pathway is configured to flow a fluid sample from the pathway input to the pathway output; a detection zone in fluidic communication with the pathway outlet; one or more reagent pads in fluidic communication with the fluidic pathway; a sample inlet port in fluidic communication with the fluidic pathway between the one or more reagent pads and the pathway outlet; and capture molecules configured to specifically bind to the analyte and disposed in the detection zone, wherein the capture molecules comprise capture molecules comprising different binding affinities for the analyte or wherein the capture molecules are disposed in the detection zone defining detection regions comprising different densities of capture molecules; a detection device configured to generate a detection zone signal based on the detection zone; and a controller operatively coupled to the detection device, the controller including logic that, when executed, causes the system to perform operations comprising generating a detection zone signal with the detection device; and generating a concentration signal based on the detection zone signal indicative of a concentration of the analyte.

In another aspect, the present disclosure provides a method for detecting a presence of an analyte in a fluid sample. In an embodiment, the method comprises flowing a fluid sample comprising the analyte into a sample inlet port in fluidic communication with a fluidic pathway comprising a pathway input and a pathway outlet; flowing a plurality of regents from one or more reagent pads through the fluidic pathway towards the pathway outlet; delivering a buffer into a buffer injection port in selective fluidic communication via an injection channel with the fluidic pathway, under conditions whereby the fluid sample and the plurality of reagents sequentially flow across capture molecules disposed in the detection zone, wherein the capture molecules comprise capture molecules comprising different binding affinities or wherein the capture molecules are disposed in the detection zone defining detection regions comprising different densities of capture molecules; and detecting the presence of the analyte bound to the plurality of detection spots, wherein a darkness of a detection spot of the plurality of detection spots is indicative of a concentration of the analyte. In an embodiment, the method is performed using an assay device or diagnostic system according to any embodiments of the present disclosure.

In an embodiment, the capture molecules comprise capture molecules comprising different binding affinities. In an embodiment, the capture molecules comprise polyclonal antibodies configured to bind to the analyte. In an embodiment, the polyclonal antibodies comprise a first antibody comprising a first binding affinity for the analyte and a second binding affinity for the analyte, and wherein the first binding affinity is different than the second binding affinity.

In an embodiment, the capture molecules are disposed in the detection zone defining detection regions comprising different densities of capture molecules, wherein the capture molecules define a plurality of detection spots disposed in the detection zone, wherein each detection spot of the plurality of detection spots comprises a density of capture molecules configured to selectively bind to the analyte. In an embodiment, a first detection spot of the plurality of detection spots comprises a first density of the capture molecules, wherein a second detection spot of the plurality of detection spots comprises a second density of the capture molecules, and wherein the first density is different than the second density. In an embodiment, each detection spot of the plurality of detection spots comprises non-capture molecules configured not to selectively bind to the analyte.

In an embodiment, a first plurality of detection spots of the plurality of detection spots defines a first row at a first lateral distance from the pathway outlet, and a second plurality of detection spots of the plurality of detection spots defines a second row at a second lateral distance from the pathway outlet, wherein the first lateral distance is different than the second lateral distance. In an embodiment, the first plurality of detection spots of the plurality of detection spots defines a first arc at a first radial distance from the pathway outlet, and the second plurality of detection spots of the plurality of detection spots defines a second arc at a second radial distance from the pathway outlet, wherein the first radial distance is different than the second radial distance. In an embodiment, a fluidic path between the pathway outlet and a detection spot of the second plurality of detection spots is between detection spots of the first plurality of detection spots.

In an embodiment, the assay device further comprises a plurality of fiducial marks disposed in the detection zone.

In an embodiment, the detection zone defines a fan-like wedge.

In an embodiment, the pathway outlet defines a constriction configured to narrow flow of the fluid sample from the fluid pathway entering the detection zone.

In an embodiment, the assay device further comprises a buffer injection port in selective fluidic communication with the fluidic pathway through an injection channel.

In an embodiment, determining the concentration of the analyte by measuring the initial rate of darkening of the plurality of detection spots.

In an embodiment, determining the concentration of the analyte by subtracting nonspecific darkening of non-capture areas of the detection zone from the darkening of the plurality of detection spots.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the present disclosure will become more readily appreciated as the same become better understood by reference to the following detailed description, when taken in conjunction with the accompanying drawings, wherein:

FIG. 1 schematically illustrates a diagnostic system, according to an embodiment of the present disclosure;

FIG. 2 schematically illustrates a detection zone of an assay device disposed in a plurality of detection spots, according to an embodiment of the present disclosure;

FIGS. 3A and 3B schematically illustrates detection of an antigen using the detection zone of FIG. 2 at various antigen concentrations, according to embodiments of the present disclosure;

FIG. 4 schematically illustrates a detection zone including a capture molecule and non-capture molecules, according to an embodiment of the present disclosure;

FIG. 5A is a perspective illustration of an assay device, according to an embodiment of the present disclosure;

FIG. 5B is a top-down plan view of the assay device of FIG. 5A, according to an embodiment of the present disclosure;

FIG. 5C schematically illustrates a detection zone of the assay device of FIG. 5A, according to an embodiment of the present disclosure;

FIG. 5D schematically illustrates another detection zone of the assay device of FIG. 5A, according to an embodiment of the present disclosure;

FIGS. 6A-6C schematically illustrates use of an assay device, according to an embodiment of the preset disclosure; and

FIG. 7 includes images of a detection zone of an assay device, according to an embodiment of the present disclosure, at various antigen concentrations.

### DETAILED DESCRIPTION

Embodiments of assay devices, diagnostic systems, and methods for analyzing an analyte, such as determining a concentration of an analyte, are described. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “selecting”, “identifying”, “capturing”, “adjusting”, “analyzing”, “determining”, “estimating”, “generating”, “comparing”, “modifying”, “receiving”, “providing”, “displaying”, “interpolating”, “outputting”, or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such as information storage, transmission, or display devices.

The algorithms and displays presented herein are not inherently related to any particular computer or other apparatus. Various general-purpose systems can be used with programs in accordance with the teachings herein, or it can prove convenient to construct a more specialized apparatus to perform the required method steps. The required structure for a variety of these systems will appear from the description below. In addition, embodiments of the present disclosure are not described with reference to any particular programming language. It will be appreciated that a variety of programming languages can be used to implement the teachings of the disclosure as described herein.

The present disclosure provides systems, devices, and methods for measuring or determining analyte concentration over a wide range of analyte concentrations in largely automated fashion and without the use of involved user intervention or complicated machinery. In this regard, the present disclosure provides devices, systems, and methods for detecting an analyte in a sample, such as a fluid sample.

Laboratory-based analyte concentration measurements are frequently performed using a class of assays generally referred to enzyme-linked immunosorbent assay (ELISA). In such assay, a sample is frequently serially diluted and then tested at various concentrations. This approach is labor intensive and requires a level of skill, equipment, and precision not practical outside of a laboratory setting.

In certain ways and in certain embodiments, the diagnostic systems, assay devices, and methods are configured to perform analyte concentration assays without the use serial dilution steps of the ELISA technique. Rather, such assays are performed on a porous membrane without manual or user-directed serial dilution.

The diagnostic systems, assay devices, and methods of the present disclosure are configured to provide a measurement of the fractional occupancy of a capture reagent on a fixed patch on a membrane or other surface.

Fractional occupancy of the capture site(s) can be determined by a number of factors. Without wishing to be bound by any particular theory, these factors can include 1) the concentration of the target antigen/analyte (Ag) in the original sample presented to the capture reagent, 2) the time that that sample is in the vicinity of the capture reagent, 3) the rate at which the analyte falls off that capture reagent during subsequent wash steps, 4) the efficiency of the association of subsequent components of the “complete stack”, which includes the detection reagent and the detection enzyme (complex), 5) the rate at which those components disassociate from the stack during the associated rinses between steps, and 6) loss of function of any of the components before the during the assay. Additionally, and without wishing to be bound by any particular theory, the local rate of darkening of the spots is proportional not only to the concentration of functional enzymes present within the capture zone, but also concentration of the chromogenic substrates (in this case both reduced DAB and H₂O₂). Through control of these factors, it is possible to measure the number of detection enzymes present within a spot on nitrocellulose as described further herein.

In an embodiment, the device comprises a fluidic pathway comprising a pathway input and a pathway outlet, wherein the fluidic pathway is configured to flow the fluid sample from the pathway input to the pathway output; a detection zone in fluidic communication with the pathway outlet; one or more reagent pads in fluidic communication with the fluidic pathway; a buffer injection port in selective fluidic communication with the fluidic pathway through an injection channel; a sample inlet port in fluidic communication with the fluidic pathway between the one or more reagent pads and the pathway outlet; and a plurality of detection spots disposed in the detection zone, wherein each detection spot of the plurality of detection spots comprises a density of capture molecules configured to selectively bind to the analyte.

### Diagnostic System

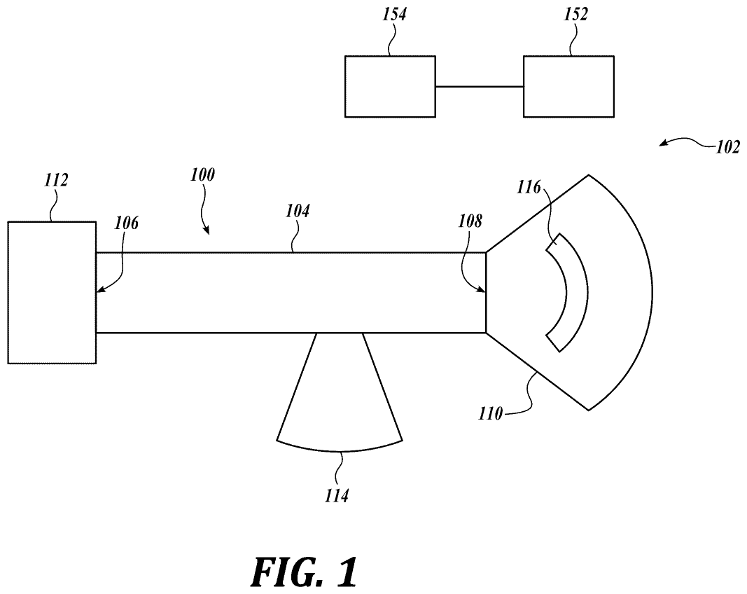

In an aspect, the present disclosure provides a diagnostic system for analyzing an analyte, such as analyzing or determining a concentration of an analyte in a sample. In this regard, attention is directed to FIG. 1 in which a diagnostic system 102, according to an embodiment of the present disclosure, is illustrated.

As shown, the diagnostic system 102 includes an assay device 100, a detection device 152, and a controller 154 operatively coupled to the detection device 152. In the illustrated embodiment, the assay device 100 includes a fluidic pathway 104 comprising a pathway input 106 and a pathway outlet 108, wherein the fluidic pathway 104 is configured to flow a fluid sample from the pathway input 106 to the pathway output, and a detection zone 110 in fluidic communication with the pathway outlet 108. The assay device 100 is shown to further include a reagent pad 112 in fluidic communication with the fluidic pathway 104.

The assay device 100 is shown to include a sample inlet port 114 in fluidic communication with the fluidic pathway 104 between the reagent pad 112 and the pathway outlet 108.

Capture molecules 116 configured to specifically bind to the analyte are shown disposed in the detection zone 110. In an embodiment, the capture molecules 116 comprise capture molecules 116 comprising different binding affinities for the analyte. In an embodiment, the capture molecules 116 are disposed in the detection zone 110 defining detection regions comprising different densities of capture molecules 116. As discussed further herein, capture molecules 116 having different analyte binding affinities or detection regions comprising different analyte binding affinities are configured to bind analyte with varying affinity and are suitable for detection of analytes at widely varying concentrations.

While capture molecules 116 are described having an affinity for an analyte, it will be understood that the diagnostic system 102 can include a detection zone 110 with capture molecules 116 having binding affinities for more than one analyte. In this regard, in an embodiment, the detection zone 110 comprises capture molecules 116 having separately binding affinities for and being configured to selectively bind to more than one analyte. In this regard, the diagnostic system 102 is configured to bind to, visualize, and measure concentrations of a plurality of analytes in a sample.

In an embodiment, the detection zone 110 comprises capture molecules 116 or other capture reagents comprising different binding affinities for the analyte, wherein the capture molecules 116 or other capture reagents are disposed in a single spot or other detection region with the detection zone 110. As described further herein, a single detection spot or zone comprising capture molecules 116 or other capture reagents comprising different binding affinities for the analyte is configured to provide indications of analyte concentration over a wide range of analyte concentration due at least in part to the varying binding affinities.

In an embodiment, the capture molecules 116 comprise polyclonal antibodies configured to bind to the analyte. In an embodiment, the polyclonal antibodies comprise a first antibody comprising a first binding affinity for the analyte and a second binding affinity for the analyte, and wherein the first binding affinity is different than the second binding affinity. In an embodiment, the polyclonal antibodies comprise antibodies with binding affinities for the analyte spanning a range of binding affinities.

A binding event, such as that between a capture reagent on a spot and the analyte, reflects a balance between the on-rate for the binding and the off-rate. For any given analyte-capture reagent binding pair, at a given set of environmental conditions, there is a specific ratio of open to full binding sites determined by the two rates, known as K_{D}. At a fixed concentration of an antigen in solution, the degree of occupancy of the capture antibody will be 50% at K_{D}. The concentration of the capture reagent is fixed, so the fractional occupancy of its binding sites varies with the concentration of the analyte in solution in a predictable manner. Without wishing to be bound by any particular theory, it is understood that a useful detection range is about ±10× around K_{D}, in that below K_{D}/10, the occupancy is below 5%, and above K_{D}×10, the occupancy is greater than 95%. Particularly at the high concentration range, it may be difficult to differentiate different concentrations because the capture reagent is effectively saturated. Below K_{D}/10, the ability to quantify may be dependent on a very sensitive detection method. One can measure smaller levels of occupancy of the capture reagent much less than 5% thereby quantifying the analyte of choice. However, a better way to measure the analyte is to have its concentration between those two extremes relative to K_{D}.

Using one spot comprising polyclonal Abs with a mean K_{D} somewhere in the middle of the range of expected concentrations of the analyte is therefore advantageous in measuring analyte concentration.

Since polyclonal Abs are a mixture of Abs with varying affinities for the target analyte against which they were produced, they therefore contain at least one higher-affinity Ab, and a range of others of higher K_{D} values. A single spot, therefore, can provide a more graded response to the analyte concentration across a wide range of analyte concentrations; at low concentrations of Ag, only the most avid Abs would saturate, but the polyclonal Abs with higher K_{D}s would be largely empty. It is therefore possible to use only one such spot and still achieve sensitivity over a wide range of analyte concentration. In an embodiment, a spot of a polyclonal does not allow as high a degree of Ab saturation (dark spots) at low concentrations of analyte as would the best high-affinity monoclonal antibodies or other high-affinity capture reagents.

While polyclonal antibodies are described, it will be understood that binding or capture chemistry with similar binding sites, but that vary with respect to their K_{D} values for an analyte, are possible and within the scope of the present disclosure.

The diagnostic system 102 is shown to include a detection device 152 configured to generate a detection zone 110 signal based on the detection zone 110. In an embodiment, the detection device 152 comprises a photodetector, such as a camera, phone, or other device configured to generate a signal based on the detection zone 110 indicative of analyte binding, such as darkness of all or a portion of the detection zone 110. In an embodiment, the detection device 152 comprises a light configured to excite detection chemistry, such as may result in fluorescence of the detection chemistry. In an embodiment, the detection device 152 is configured to generate a black and white or grayscale image of the detection zone 110.

As above, the diagnostic system 102 includes a controller 154 operatively coupled to the detection device 152. In an embodiment, the controller 154 includes logic that, when executed, causes the system 102 to perform operations. Such operations can include one or more or all operations, steps, or processes for performing methods of the present disclosure, discussed further herein. In an embodiment, the operations include generating a detection zone 110 signal with the detection device 152; and generating a concentration signal based on the detection zone 110 signal indicative of a concentration of the analyte.

In an embodiment, the controller 154 is configured to convey an image stack, such as generated by the detection device 152, to a remote site where the images could be interpreted such as by software on a centralized computing facility to produce concentration values from the image stack. In an embodiment, those values could be sent back to the diagnostic system 102, such as to be displayed to the user, or sent to the user's cell phone, or sent back only in interpreted form without access to raw numbers.

In its most basic configuration, the controller 154 includes at least one processor and a system memory connected by a communication bus. Depending on the exact configuration and type of device, system memory may be volatile or nonvolatile memory, such as read only memory (“ROM”), random access memory (“RAM”), EEPROM, flash memory, or similar memory technology. Those of ordinary skill in the art will recognize that system memory typically stores data and/or program modules that are immediately accessible to and/or currently being operated on by the processor. In this regard, the processor may serve as a computational center of controller 154 by supporting the execution of instructions.

The controller 154 may include a network interface comprising one or more components for communicating with other devices over a network. Embodiments of the present disclosure may access basic services that utilize network interface to perform communications using common network protocols. Network interface may also include a wireless network interface configured to communicate via one or more wireless communication protocols, such as WiFi, 2G, 3G, 4G, LTE, WiMAX, Bluetooth, and/or the like.

In an embodiment, the controller 154 also includes a storage medium. However, services may be accessed using a computing device that does not include means for persisting data to a local storage medium. Therefore, the storage medium may be omitted. In any event, the storage medium may be volatile or nonvolatile, removable or nonremovable, implemented using any technology capable of storing information such as, but not limited to, a hard drive, solid state drive, CD-ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer-readable instructions, data structures, program modules, or other data.

Suitable implementations of computing devices that include a controller 154, system memory, communication bus, storage medium, and network interface are known and commercially available. For ease of illustration and because it is not important for an understanding of the claimed subject matter, FIG. 1 does not show some of the typical components of many computing devices. In this regard, the controller 154 may include input devices, such as a keyboard, keypad, mouse, microphone, touch input device, touch screen, tablet, and/or the like. Such input devices may be coupled to controller 154 by wired or wireless connections including RF, infrared, serial, parallel, Bluetooth, USB, or other suitable connection protocols using wireless or physical connections. Since these devices are well known in the art, they are not illustrated or described further herein.

The processes and user-interface described above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, some of the processes or logic for implementing the user-interface may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

### Assay Device

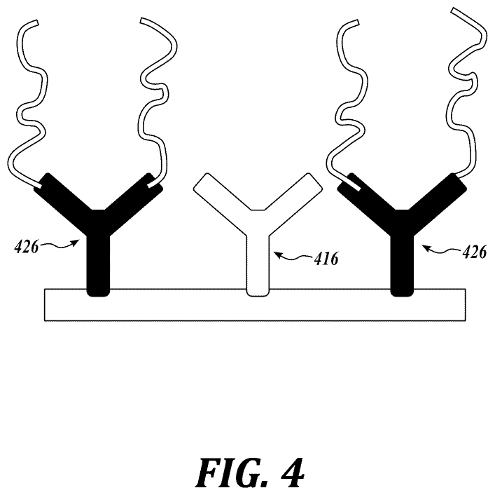

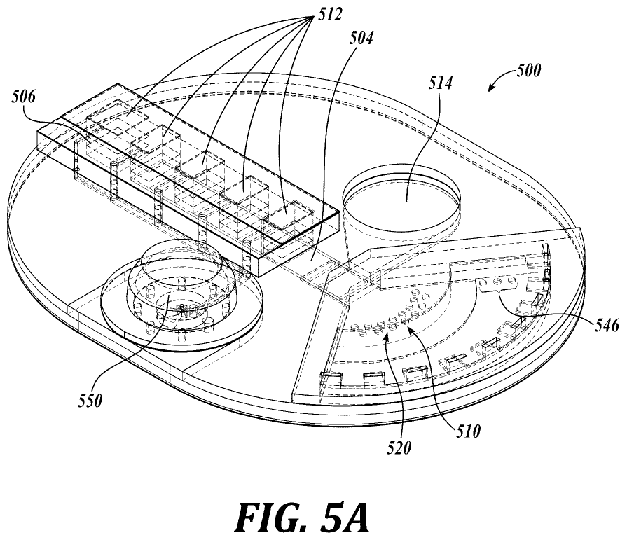

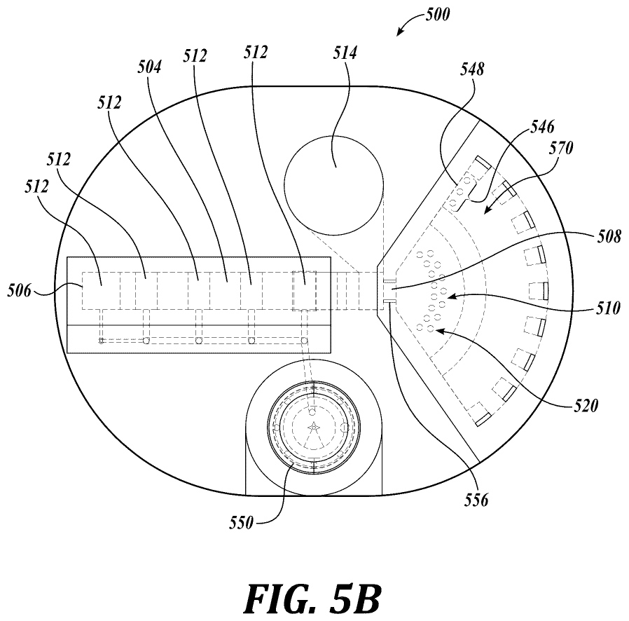

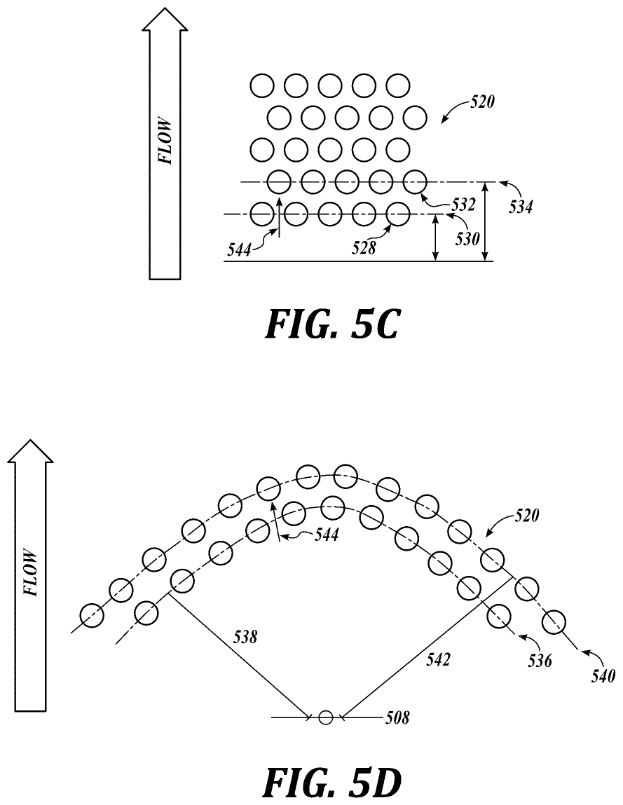

In another aspect, the present disclosure provides an assay device for detection of an analyte in a fluid sample. In this regard, attention is directed to FIGS. 5A-5D in which an assay device 500, according to an embodiment of the present disclosure, is illustrated. FIG. 5A is a perspective illustration of the assay device 500. FIG. 5B is a top-down plan view of the assay device 500. FIG. 5C schematically illustrates a detection zone 510 of the assay device 500. FIG. 5D schematically illustrates another detection zone 510 of the assay device 500 of FIG. 5A. In an embodiment, the assay device 500 is an example of the assay device 100 described further herein with respect to FIG. 1.

In the illustrated embodiment, the assay device 500 is shown to include a fluidic pathway 504, a detection zone 510, reagent pads 512, a sample port inlet, and capture molecules disposed in the detection zone 510. As shown, the fluidic pathway 504 comprises a pathway input 506 shown disposed at a first or upstream end of the fluidic pathway 504 and a pathway outlet 508 shown disposed at a second of downstream end of the fluidic pathway 504. In an embodiment, the fluidic pathway 504 is configured to flow the fluid sample from the pathway input 506 to the pathway output. In an embodiment, the fluidic pathway 504 comprises one or more porous materials, such as a porous pad, membrane or wick, configured to flow the fluid sample through capillary action. Representative examples of such porous membranes include paper, nitrocellulose, nylon, glass wool, and many other materials recognized by those skilled in the art as capable of serving as a wick in the context of the present technology. The wick can be two-dimensional or three-dimensional (when considering its height in addition to its length and width). In some embodiments, the wick is a single layer, while in other embodiments, the wick comprises two or more layers of membrane. Because the fluidic pathway 504 is membranous, fluid traverses the pathway via capillary action or wicking. The width of the pathway is defined by sides or edges that limit the area of the pathway that can be traversed by fluid. The fluidic pathway 504 can be patterned on a wick either by cutting the wick or by deposition of an insoluble barrier to create the desired configuration of pathways and pathway intersection(s).

As above, the assay device 500 also includes a detection zone 510 in fluidic communication with the pathway outlet 508. In an embodiment, the assay device 500 comprises capture molecules configured to selectively bind to the analyte and disposed in the detection zone 510. In an embodiment, the capture molecules comprise capture molecules comprising different binding affinities for the analyte or wherein the capture molecules are disposed in the detection zone 510 defining detection regions comprising different densities of capture molecules.

The assay device 500 comprises capture molecules configured to selectively bind to the analyte and disposed in the detection zone 510. As described further herein, in an embodiment, the capture molecules comprise capture molecules comprising different binding affinities for the analyte. As also described further herein, in an embodiment, the capture molecules are disposed in the detection zone 510 defining detection regions comprising different densities of capture molecules.

In an embodiment, the capture molecules are disposed in the detection zone 510 defining detection regions comprising different densities of capture molecules. In an embodiment, the capture molecules are disposed in the detection zone 510 to define a plurality of detection spots 520, wherein each detection spot of the plurality of detection spots 520 comprises a density of capture molecules configured to selectively bind to the analyte.

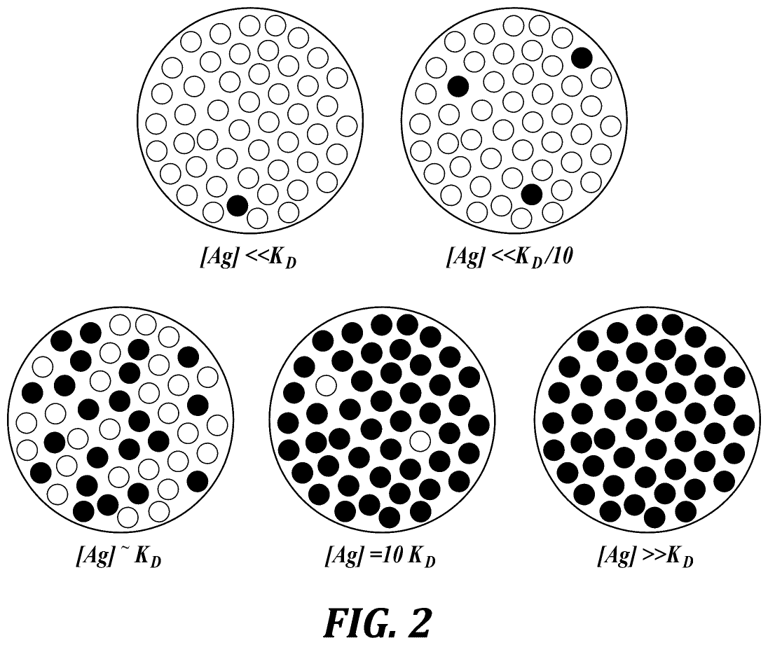

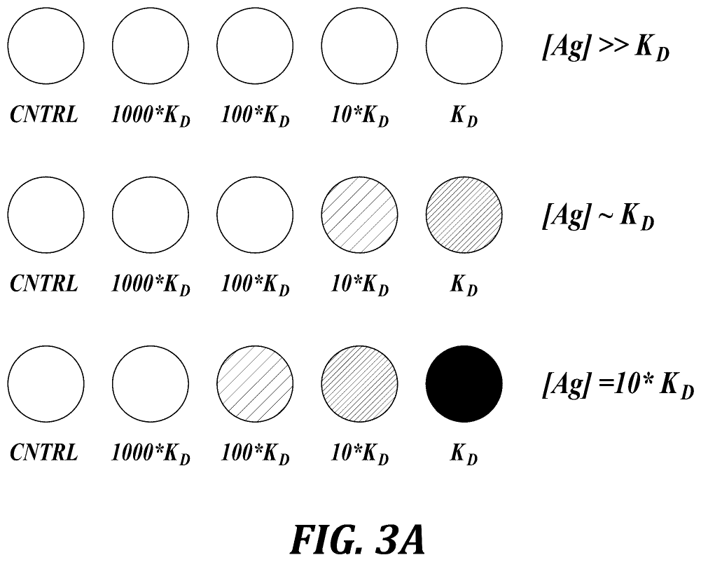

In this regard, attention is directed to FIG. 2 in which a schematic illustration of a detection zone of an assay device according to an embodiment of the present disclosure, is illustrated. Attention is also directed to FIGS. 3A and 3B which schematically illustrate detection of an antigen using the detection zone of FIG. 2 at various antigen or analyte concentrations. In an embodiment, the detection zone of FIGS. 2 and 3A and 3B are examples of the detection zone 510 of assay device 500 discussed further herein with respect to FIGS. 5A-5D.

As shown, the detection zone comprises capture reagents disposed in the form of a plurality of spots. In this regard, the detection zone comprises a first detection spot of the plurality of detection spots comprises a first density of the capture molecules. Further, a second detection spot of the plurality of detection spots comprises a second density of the capture molecules, and wherein the first density is different than the second density. As shown, detection spots of the plurality of detection spots comprise varying densities of capture molecules corresponding to a range of K_{D}.

In an embodiment, the capture molecules comprise monoclonal antibodies, such as monoclonal antibodies having a high binding affinity for the analyte.

In an embodiment, antibody occupancy (dark circles) with antigen is expected on either side of K_{D} within a single capture spot independent of detection mechanism. Without wishing to be bound by any particular theory, it is understood that 50% occupancy of capture molecules occurs when the analyte concentration is equal to K_{D}. At ten times below K_{D} very few capture molecules will be occupied. In this regard, it may be difficult to distinguish that from any lower analyte concentration for which the capture molecules will be nearly empty. Conversely, at ten times above K_{D}, very few capture molecules will be unoccupied. In such a scenario, it may be difficult to distinguish anything higher from saturation of all the capture molecules. A single spot with capture molecules at appropriate K_{D} would be adequate for quantification of analyte concentration as long as that concentration was approximately between K_{D}/5 and 5K_{D}.

In an embodiment, the plurality of detection spots comprises detections spots configured to quantify analyte that varies more than 10-fold. To quantify the analyte concentration, multiple capture spots comprise capture molecules comprising progressively degraded affinities (higher K_{D} values). Shown are expected degrees of spot occupancy for the analyte over a range of analyte concentration using capture molecules with degraded affinities in steps of 10. If analyte concentration were much smaller than K_{D}, the right-most spot would be colorless. By measuring which spots are in the intermediate occupancy levels, it is possible to quantify the concentration of concentration at concentrations higher than K_{D}. By increasing the number of spots and the degree of degradation, it is possible to achieve finer definition of the analyte concentration.

As above, in conventional ELISA “bringing the analyte into range” is accomplished by performing a series of serial dilutions of the sample. This is possible in paper microfluidics, but presents challenges that can be avoided in the following way. In the present disclosure, a sample is brought to the plurality of detection spots, each of which was covered with capture molecules that had a different molecular value of KD. For example, to measure the concentration of the analyte over a range of 1000 (which can be useful to monitor some medical conditions), the assay device comprises, for example, 4 spots: Spot 1 comprises molecules with the lowest K_{D}, and would be able to be half-saturated at analyte concentration of ~K_{D}. That spot could be used to measure analyte concentration down to at least K_{D}/10. It could also be able to measure the analyte concentration at concentrations almost 10× higher, but above that concentration the spot would saturate. To measure higher concentrations, the next spot could comprise an Ab (or equivalent binding entity) with a K_{D} between 10 and 100 times higher (i.e., less sensitive). And another with an even higher K_{D}. And finally, a 4th spot covered with capture molecules with no known affinity for the Ag at all, to provide a “control” for nonspecific binding.

In other words, in an embodiment, the present disclosure provides assay devices configured to quantitatively measure the concentration of an Ag by presenting the sample with a plurality of capture spots, each of which have binding molecules with the same specificity, but sensitivities (as quantified by K_{D}s) that span a range of the anticipated range of analyte concentration anticipated to be found in the sample. In this regard, the goal is to have one or two of the spots have an occupancy level measured as gray (neither empty nor saturated), while at least one spot will be blank and, if a sufficiently low K_{D} binder were available, another spot in the set would be saturated. By measuring the degree of saturation of the set of spots, such as in comparison to a set of calibration spots or other fiducial markers with known concentrations of the detection enzymes, it is possible to take the darkening rates of the sample spots and using interpolation, determine the concentration of the analyte in question.

While high-affinity, monoclonal antibodies are described, it will be understood that binders of varying analyte binding affinity are possible and within the scope of the present disclosure. Such binders of varying analyte binding affinity can include de novo designed proteins, such as those formed using a RFdiffusion method. One could use this method to create binding molecules to analytes of interest that have defined affinities (and K_{D}s) for the target analytes, and use the binders as capture molecules. These binders may or may not resemble antibody Fab fragments. In an embodiment, the binders comprise not only high-affinity binders, but also binders with a range of lower affinity for the analyte that could be made or modified into molecules with high affinity for NC membranes and spotted thereupon.

In an embodiment, the capture molecules include aptamers, such as aptamers having an affinity for the analyte. In an embodiment, the capture molecules comprise a plurality of aptamers, such as a plurality of aptamers comprising varying affinities for an analyte.

In an embodiment, each detection spot of the plurality of detection spots comprises non-capture molecules configured not to selectively bind to the analyte. These are schematically illustrated, for example, in FIG. 2 as white dots.

In an embodiment, the non-capture molecules comprise bovine serum albumin (BSA) or another molecule configured to prevent or limit non-specific adhesion of the analyte or other sample molecule to a substrate in the detection zone.

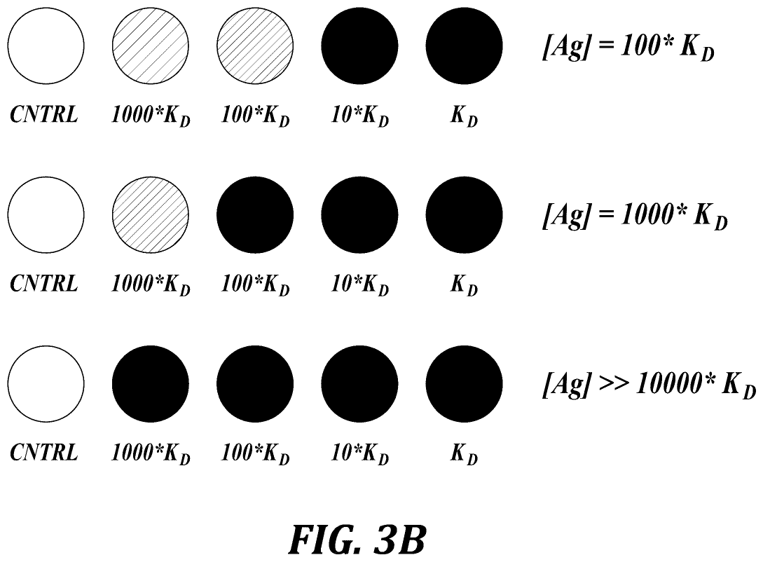

FIG. 4 schematically illustrates a detection zone including a capture molecule 416 and non-capture molecules 426, according to an embodiment of the present disclosure, which will now be discussed. In an embodiment, the detection zone is an example of the detection zone described further herein with respect to FIGS. 2 and 3A and 3B, or an example of the detection zone 510 discussed further herein with respect to FIGS. 5A-5D.

As shown, the detection zone comprises a high-affinity monoclonal antibody 416 against an Ag. The high-affinity monoclonal antibody 416 is surrounded with molecules 426 that limit or inhibit rapid or long-lasting binding. In this regard, it is possible to selectively and in a controlled manner increase an effective K_{D} of the detection zone at the molecular level. In the illustrated embodiment, the non-capture molecules 426 comprise poly(ethylene glycol) (PEG) chains coupled to an antibody configured to bind to the PEG chains. In an embodiment, a mixture of the desired capture Ab 416 with another non-capture Ab 426 that binds to (or is prebound to) a PEG chain is co-adsorbed onto the detection zone. In an embodiment, the non-capture molecules 426 comprise Abs specific to biotin, which are coupled to PEG chains of lengths ranging for an n of 2 to an n of 36, which further comprise a streptavidin molecule.

In an embodiment, different spots of a detection zone have the biotinylated PEG chains of different lengths, such as where longer PEG chains are configured to provide a larger depressive effect on the capture Ab's affinity for the selected analyte. The addition of the PEG chains to the anti-PEG Abs can occur before or after spotting. In an embodiment, the detection zone comprises a series of spots comprising, for example, the same ratio of capture Ab 416 to non-capture/anti-biotin Ab 426, but with different lengths of PEG on each spot. In an embodiment, to prevent possible biotin in a biological sample from displacing the PEG chains from the anti-PEG Abs, free biotin is immobilized upstream of the detection zone with biotin capture spots.

Referring again to FIGS. 5C and 5D, embodiments of pluralities of detection spots will now be described with respect arrangements of spotted capture reagents. Lines are commonly used in a lateral flow strip assay, in part because commercial striping systems lead to the least expensive final products. The stripers allow one or more lines to printed onto large NC sheets, that are then cut perpendicular to the capture stripes into narrow individual strips. However, there are problems getting high densities of capture stripes onto strips. Spot arrays, according to embodiments of the present disclosure, minimize or reduce the use of capture reagents, and provide other advantages with respect to shielding, as described further herein.

A single horizontal line of spots across the flow path is possible. However, such an arrangement would require a very wide detection zone 510, which would use large amounts of reagents and would possibly result in the lack of binding by the low-concentration analytes. In an embodiment, upstream paper fluidics (i.e., the fluidic pathway 504) terminate and pass through a narrow porous membrane or constriction 556. As shown, the detection zone 510, disposed adjacent to and downstream of the fluidic pathway 504, which comprises the capture spots, defines a widening arc of porous membrane or substrate. Such a structure is configured to allow relatively rapid upstream flow, and give uniform flows across the porous membrane “pie wedge” 548. By combining aspects of the wedge shape 548 with a tight non-overlapping flow field for the spots, good analyte binding is provided.

FIG. 5C schematically illustrates a detection zone 510 of the assay device 500 of FIG. 5A, according to an embodiment of the present disclosure. As shown, a first plurality of detection spots of the plurality of detection spots 520 defines a first row 528 at a first lateral distance 530 from the pathway outlet 508, and a second plurality of detection spots of the plurality of detection spots 520 defines a second row 532 at a second lateral distance 534 from the pathway outlet 508. In the illustrated embodiment, the first lateral distance 530 is different than the second lateral distance 534. In this regard, the first plurality of detection spots is disposed upstream of the second plurality of detection spots.

As shown, the second plurality of detection spots is offset from the first plurality of detection spots 520. In the illustrated embodiment, a fluidic path 544 between the pathway outlet 508 and a detection spot of the second plurality of detection spots is between detection spots of the first plurality of detection spots. In this way, upstream spots do not shield, or shield less, downstream spots from reagents and analyte, thereby allowing more reagents and analyte to reach the downstream spots.

FIG. 5D schematically illustrates another detection zone 510 of the assay device 500 of FIG. 5A, according to an embodiment of the present disclosure. As shown, the first plurality of detection spots of the plurality of detection spots 520 defines a first arc 536 at a first radial distance 538 from the pathway outlet 508, and the second plurality of detection spots of the plurality of detection spots 520 defines a second arc 540 at a second radial distance 542 from the pathway outlet 508. In the illustrated embodiment, the first radial distance 538 is different than the second radial distance 542. In this regard, the first plurality of detection spots is upstream of the second plurality of detection spots.

In the illustrated embodiment, a fluidic path 544 between the pathway outlet 508 and a detection spot of the second plurality of detection spots is between detection spots of the first plurality of detection spots. In this way, upstream spots do not shield, or shield less, downstream spots from reagents and analyte, thereby allowing more reagents and analyte to reach the downstream spots.

As shown, the assay device 500 comprises one or more reagent pads 512 in fluidic communication with the fluidic pathway 504. In an embodiment, the one or more reagents pads comprise reagents for a detection reaction and/or processing the sample. In an embodiment, the reagent pads 512 comprise reagents configured to selectively bind to the analyte, such as in a sandwich immunoassay. In an embodiment, the reagent pads 512 comprise reagents configured to visualize the analyte, such as when immobilized in the detection zone 510. In an embodiment, the reagents in the one or more reagents pads are lyophilized, dried, or otherwise immobilized on the reagent pads 512 such that they may be dissolved when contacted with a buffer or other liquid in use of the assay device 500.

In the illustrated embodiment, the assay device 500 comprises a sample inlet port 514 in fluidic communication with the fluidic pathway 504 between the one or more reagent pads 512 and the pathway outlet 508. In an embodiment, the sample inlet port 514 is configured to receive a sample, such as a biological sample, and transport at least a portion of the sample to the fluidic pathway 504 and eventually the detection zone 510. In an embodiment, the sample inlet port 514 is configured to receive a biological sample selected from whole blood, blood plasma, urine, saliva, mucus, feces, and the like.

As shown, the assay device 500 comprises a buffer injection port 550 in selective fluidic communication with the fluidic pathway 504 through an injection channel. In an embodiment, the buffer injection port 550 is configured to deliver buffer to the fluidic pathway 504 through actuation of the buffer injection port 550, such as by a user. In the illustrated embodiment, the buffer injection port 550 is configured to deliver buffer to the fluidic pathway 504 downstream of at least one of the reagent pads 512, although it will be understood that the buffer injection port 550 can be otherwise configured to deliver buffer to any portion of the fluidic pathway 504.

The assay device 500 is also shown to include a plurality of fiducial marks 546 disposed in the detection zone 510. Such fiducial marks 546 can include marks indicating darkness or other color corresponding to bound analyte at various concentrations, thus providing further information regarding or confirmation of analyte concentration in the sample.

In an embodiment, the pathway outlet 508 defines a constriction 556 configured to narrow flow of the fluid sample from the fluidic pathway 504 entering the detection zone 510.

### Method

In another aspect, the present disclosure provides a method, such as for detecting a presence of or a concentration of an analyte in a fluid sample. In an embodiment, the method is suitable to be performed on an assay device or a diagnostic system according to various aspects of the present disclosure, such as the assay device 500 discussed further herein with respect to FIGS. 2-4 and 5A-5D and the diagnostic system 102 discussed further herein with respect to FIG. 1.

In an embodiment, the method can begin with flowing a fluid sample comprising the analyte into a sample inlet port in fluidic communication with a fluidic pathway comprising a pathway input and a pathway outlet. In an embodiment, the fluid sample flows into the fluidic pathway through the pathway input and to the pathway outlet. In an embodiment, such flow of the fluid sample is through capillary action of the fluid sample through the fluidic pathway, which, in some embodiments, comprises a porous membrane or other bibulous network.

In an embodiment, the method further comprises flowing a plurality of reagents from one or more reagent pads, such as one or more reagent pads disposed in the fluidic pathway, through the fluidic pathway towards the pathway outlet. In this regard, the reagents are transported from an upstream portion of the fluidic pathway downstream of the one or more reagent pads.

In an embodiment, the method further comprises delivering a buffer from a buffer injection port in selective fluidic communication via an injection channel with the fluidic pathway. In an embodiment, the fluid sample and the plurality of reagents thereby sequentially flow across capture molecules disposed in a detection zone. In an embodiment, the capture molecules comprise capture molecules comprising different binding affinities or wherein the capture molecules are disposed in the detection zone defining detection regions comprising different densities of capture molecules, as discussed further herein with respect to FIGS. 1-4 and 5A-5D.

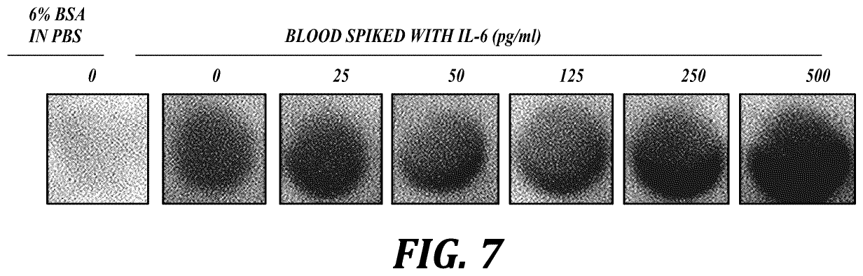

In an embodiment, the method further comprises detecting, such as with a detection device, the presence of the analyte bound to the plurality of detection spots. In an embodiment, a darkness of a detection spot of the plurality of detection spots is indicative of a concentration of the analyte bound thereto. See for example FIG. 7.

In an embodiment, the method further comprises determining a concentration of the analyte in the sample. In an embodiment, determining the concentration of the analyte comprises measuring the initial rate of darkening of the plurality of detection spots. In an embodiment, determining the concentration of the analyte comprises subtracting nonspecific darkening of non-capture areas of the detection zone from the darkening of the plurality of detection spots.

In an embodiment, determining a concentration of an analyte in the sample comprises comparing darknesses of detection spots. In this regard, the comparison can comprise determining which spots are entirely dark (i.e., saturated with bound analyte) and which spots are not saturated with bound analyte, and determining a density of capture molecules in the detection spots. As discussed further herein, detection spots comprising different densities of capture molecules will saturate with bound analyte at different concentrations. Further, partial occupancy of a detection spot comprising a K_{D} will indicate a concentration of the analyte in the sample.

Determining a concentration of the analyte can also comprise comparing a darkness of one or more detection spots with fiducial markers on an assay device, such as fiducial markers indicating a correspondence between darkness and concentration of the analyte.

It is appreciated that the order in which some or all of the method steps should not be deemed limiting. Rather, one of ordinary skill in the art having the benefit of the present disclosure will understand that some of the method steps may be executed in a variety of orders not illustrated, or even in parallel.

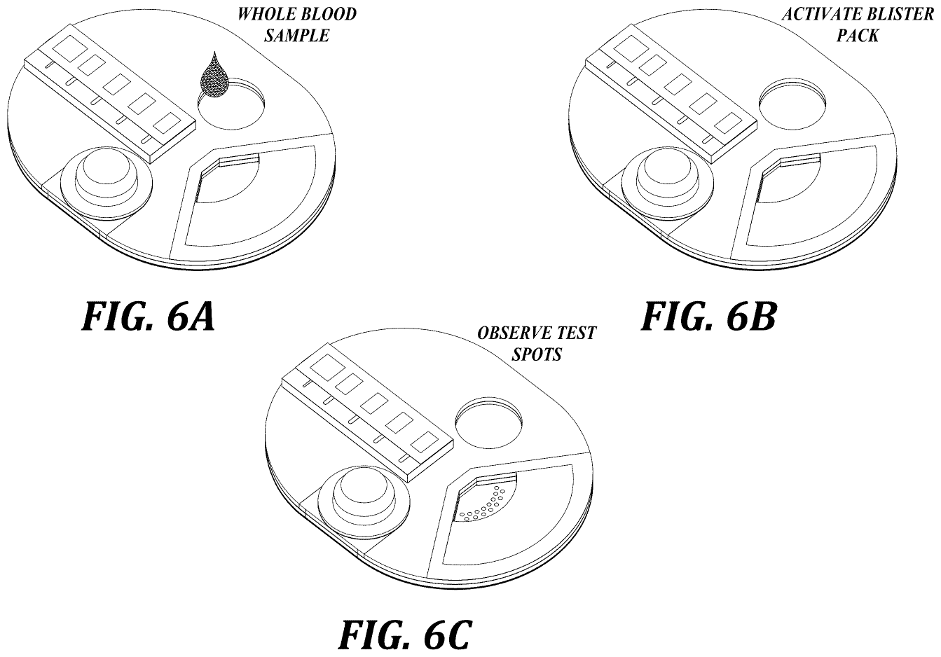

An example method according to an embodiment of the present disclosure will now be described with respect to FIGS. 6A-6C. As shown, the method begins with providing a sample, here whole blood, to a sample inlet port. As discussed further herein, the sample is transported, such as through capillary action, to a fluidic pathway and on to a detection zone.

Next the method includes activating a blister pack, such as by puncturing the blister pack through actuation thereof, thereby dissolving and subsequently transporting one or more reagents to the detection zone and over analyte bound to the detection zone.

As shown, the method next includes observing test or detection spots. As discussed further herein, a darkness of the detection spots can be indicative of a concentration of an analyte in the sample.

Embodiments of the present disclosure include:
1. An assay device for detection of an analyte in a fluid sample, the assay device comprising:
   a fluidic pathway comprising a pathway input and a pathway outlet, wherein the fluidic pathway is configured to flow the fluid sample from the pathway input to the pathway output;
   a detection zone in fluidic communication with the pathway outlet;
   one or more reagent pads in fluidic communication with the fluidic pathway;
   a sample inlet port in fluidic communication with the fluidic pathway between the one or more reagent pads and the pathway outlet; and capture molecules configured to selectively bind to the analyte and disposed in the detection zone, wherein the capture molecules comprise capture molecules comprising different binding affinities for the analyte or wherein the capture molecules are disposed in the detection zone defining detection regions comprising different densities of capture molecules.
2. The assay device of Embodiment 1, wherein the capture molecules comprise capture molecules comprising different binding affinities, wherein the capture molecules comprise polyclonal antibodies configured to bind to the analyte.
3. The assay device of Embodiment 2, wherein the polyclonal antibodies comprise a first antibody comprising a first binding affinity for the analyte and a second binding affinity for the analyte, and wherein the first binding affinity is different than the second binding affinity.
4. The assay device of Embodiment 1, wherein the capture molecules are disposed in the detection zone defining detection regions comprising different densities of capture molecules, wherein the capture molecules define a plurality of detection spots disposed in the detection zone, wherein each detection spot of the plurality of detection spots comprises a density of capture molecules configured to selectively bind to the analyte.
5. The assay device of Embodiment 4, wherein a first detection spot of the plurality of detection spots comprises a first density of the capture molecules, wherein a second detection spot of the plurality of detection spots comprises a second density of the capture molecules, and wherein the first density is different than the second density.
6. The assay device of Embodiment 5, wherein each detection spot of the plurality of detection spots comprises non-capture molecules configured not to selectively bind to the analyte.
7. The assay device of Embodiment 5, wherein a first plurality of detection spots of the plurality of detection spots defines a first row at a first lateral distance from the pathway outlet, and a second plurality of detection spots of the plurality of detection spots defines a second row at a second lateral distance from the pathway outlet, wherein the first lateral distance is different than the second lateral distance.
8. The assay device of Embodiment 7, wherein the first plurality of detection spots of the plurality of detection spots defines a first arc at a first radial distance from the pathway outlet, and the second plurality of detection spots of the plurality of detection spots defines a second arc at a second radial distance from the pathway outlet, wherein the first radial distance is different than the second radial distance.
9. The assay device of Embodiment 7, wherein a fluidic path between the pathway outlet and a detection spot of the second plurality of detection spots is between detection spots of the first plurality of detection spots.
10. The assay device of Embodiment 1, further comprising a plurality of fiducial marks disposed in the detection zone.
11. The assay device of Embodiment 1, wherein the detection zone defines a fan-like wedge.
12. The assay device of Embodiment 1, wherein the pathway outlet defines a constriction configured to narrow flow of the fluid sample from the fluid pathway entering the detection zone.
13. The assay device of Embodiment 1, further comprising a buffer injection port in selective fluidic communication with the fluidic pathway through an injection channel.
14. A diagnostic system comprising:
   an assay device comprising:
   a fluidic pathway comprising a pathway input and a pathway outlet, wherein the fluidic pathway is configured to flow a fluid sample from the pathway input to the pathway output;
   a detection zone in fluidic communication with the pathway outlet;
   one or more reagent pads in fluidic communication with the fluidic pathway;
   a sample inlet port in fluidic communication with the fluidic pathway between the one or more reagent pads and the pathway outlet; and capture molecules configured to specifically bind to the analyte and disposed in the detection zone, wherein the capture molecules comprise capture molecules comprising different binding affinities for the analyte or wherein the capture molecules are disposed in the detection zone defining detection regions comprising different densities of capture molecules;
   a detection device configured to generate a detection zone signal based on the detection zone; and a controller operatively coupled to the detection device, the controller including logic that, when executed, causes the system to perform operations comprising:
   generating a detection zone signal with the detection device; and generating a concentration signal based on the detection zone signal indicative of a concentration of the analyte.
15. The assay diagnostic system of Embodiment 14, wherein the capture molecules comprise capture molecules comprising different binding affinities, wherein the capture molecules comprise polyclonal antibodies configured to bind to the analyte, wherein the polyclonal antibodies comprise a first antibody comprising a first binding affinity for the analyte and a second binding affinity for the analyte, and wherein the first binding affinity is different than the second binding affinity.
16. The assay diagnostic system of Embodiment 14, further comprising a plurality of detection spots disposed in the detection zone, wherein each detection spot of the plurality of detection spots comprises a density of capture molecules configured to selectively bind to the analyte.
17. The assay diagnostic system of Embodiment 16, wherein a first detection spot of the plurality of detection spots comprises a first density of the capture molecules, wherein a second detection spot of the plurality of detection spots comprises a second density of the capture molecules, and wherein the first density is different than the second density.
18. A method for detecting a presence of an analyte in a fluid sample, the method comprising:
   flowing a fluid sample comprising the analyte into a sample inlet port in fluidic communication with a fluidic pathway comprising a pathway input and a pathway outlet;
   flowing a plurality of regents from one or more reagent pads through the fluidic pathway towards the pathway outlet;
   delivering a buffer into a buffer injection port in selective fluidic communication via an injection channel with the fluidic pathway, under conditions whereby the fluid sample and the plurality of reagents sequentially flow across capture molecules disposed in the detection zone, wherein the capture molecules comprise capture molecules comprising different binding affinities or wherein the capture molecules are disposed in the detection zone defining detection regions comprising different densities of capture molecules; and
   detecting the presence of the analyte bound to the plurality of detection spots, wherein a darkness of a detection spot of the plurality of detection spots is indicative of a concentration of the analyte.
19. The method of Embodiment 18, determining the concentration of the analyte by measuring the initial rate of darkening of the plurality of detection spots.
20. The method of Embodiment 18, determining the concentration of the analyte by subtracting nonspecific darkening of non-capture areas of the detection zone from the darkening of the plurality of detection spots.

The above description of illustrated embodiments of the disclosure, including what is described in the Abstract, is not intended to be exhaustive or to limit the disclosure to the precise forms disclosed. While specific embodiments of, and examples for, the disclosure are described herein for illustrative purposes, various modifications are possible within the scope of the disclosure, as those skilled in the relevant art will recognize.

These modifications can be made to the disclosure in light of the above detailed description. The terms used in the following claims should not be construed to limit the disclosure to the specific embodiments disclosed in the specification. Rather, the scope of the disclosure is to be determined entirely by the following claims, which are to be construed in accordance with established doctrines of claim interpretation.

The embodiments of the invention in which an exclusive property or privilege is claimed are defined as follows:

1. An assay device for detection of an analyte in a fluid sample, the assay device comprising:
a fluidic pathway comprising a pathway input and a pathway outlet, wherein the fluidic pathway is configured to flow the fluid sample from the pathway input to the pathway output;
a detection zone in fluidic communication with the pathway outlet;
one or more reagent pads in fluidic communication with the fluidic pathway;
a sample inlet port in fluidic communication with the fluidic pathway between the one or more reagent pads and the pathway outlet; and
capture molecules configured to selectively bind to the analyte and disposed in the detection zone, wherein the capture molecules comprise capture molecules comprising different binding affinities for the analyte or wherein the capture molecules are disposed in the detection zone defining detection regions comprising different densities of capture molecules.

2. The assay device of claim 1, wherein the capture molecules comprise capture molecules comprising different binding affinities, wherein the capture molecules comprise polyclonal antibodies configured to bind to the analyte.

3. The assay device of claim 2, wherein the polyclonal antibodies comprise a first antibody comprising a first binding affinity for the analyte and a second binding affinity for the analyte, and wherein the first binding affinity is different than the second binding affinity.

4. The assay device of claim 1, wherein the capture molecules are disposed in the detection zone defining detection regions comprising different densities of capture molecules, wherein the capture molecules define a plurality of detection spots disposed in the detection zone, wherein each detection spot of the plurality of detection spots comprises a density of capture molecules configured to selectively bind to the analyte.

5. The assay device of claim 4, wherein a first detection spot of the plurality of detection spots comprises a first density of the capture molecules, wherein a second detection spot of the plurality of detection spots comprises a second density of the capture molecules, and wherein the first density is different than the second density.

6. The assay device of claim 5, wherein each detection spot of the plurality of detection spots comprises non-capture molecules configured not to selectively bind to the analyte.

7. The assay device of claim 5, wherein a first plurality of detection spots of the plurality of detection spots defines a first row at a first lateral distance from the pathway outlet, and a second plurality of detection spots of the plurality of detection spots defines a second row at a second lateral distance from the pathway outlet, wherein the first lateral distance is different than the second lateral distance.

8. The assay device of claim 7, wherein the first plurality of detection spots of the plurality of detection spots defines a first arc at a first radial distance from the pathway outlet, and the second plurality of detection spots of the plurality of detection spots defines a second arc at a second radial distance from the pathway outlet, wherein the first radial distance is different than the second radial distance.

9. The assay device of claim 7, wherein a fluidic path between the pathway outlet and a detection spot of the second plurality of detection spots is between detection spots of the first plurality of detection spots.

10. The assay device of claim 1, further comprising a plurality of fiducial marks disposed in the detection zone.

11. The assay device of claim 1, wherein the detection zone defines a fan-like wedge.

12. The assay device of claim 1, wherein the pathway outlet defines a constriction configured to narrow flow of the fluid sample from the fluidic pathway entering the detection zone.

13. The assay device of claim 1, further comprising a buffer injection port in selective fluidic communication with the fluidic pathway through an injection channel.

14. A diagnostic system comprising:
an assay device comprising:
a fluidic pathway comprising a pathway input and a pathway outlet, wherein the fluidic pathway is configured to flow a fluid sample from the pathway input to the pathway output;
a detection zone in fluidic communication with the pathway outlet;
one or more reagent pads in fluidic communication with the fluidic pathway;
a sample inlet port in fluidic communication with the fluidic pathway between the one or more reagent pads and the pathway outlet; and
capture molecules configured to specifically bind to the analyte and disposed in the detection zone, wherein the capture molecules comprise capture molecules comprising different binding affinities for the analyte or wherein the capture molecules are disposed in the detection zone defining detection regions comprising different densities of capture molecules;
a detection device configured to generate a detection zone signal based on the detection zone; and
a controller operatively coupled to the detection device, the controller including logic that, when executed, causes the system to perform operations comprising:
generating a detection zone signal with the detection device; and
generating a concentration signal based on the detection zone signal indicative of a concentration of the analyte.

15. The assay diagnostic system of claim 14, wherein the capture molecules comprise capture molecules comprising different binding affinities, wherein the capture molecules comprise polyclonal antibodies configured to bind to the analyte, wherein the polyclonal antibodies comprise a first antibody comprising a first binding affinity for the analyte and a second binding affinity for the analyte, and wherein the first binding affinity is different than the second binding affinity.

16. The assay diagnostic system of claim 14, further comprising a plurality of detection spots disposed in the detection zone, wherein each detection spot of the plurality of detection spots comprises a density of capture molecules configured to selectively bind to the analyte.

17. The assay diagnostic system of claim 16, wherein a first detection spot of the plurality of detection spots comprises a first density of the capture molecules, wherein a second detection spot of the plurality of detection spots comprises a second density of the capture molecules, and wherein the first density is different than the second density.

18. A method for detecting a presence of an analyte in a fluid sample, the method comprising:
flowing a fluid sample comprising the analyte into a sample inlet port in fluidic communication with a fluidic pathway comprising a pathway input and a pathway outlet;
flowing a plurality of regents from one or more reagent pads through the fluidic pathway towards the pathway outlet;
delivering a buffer into a buffer injection port in selective fluidic communication via an injection channel with the fluidic pathway, under conditions whereby the fluid sample and the plurality of reagents sequentially flow across capture molecules disposed in a detection zone, wherein the capture molecules comprise capture molecules comprising different binding affinities or wherein the capture molecules are disposed in the detection zone defining detection regions comprising different densities of capture molecules; and
detecting the presence of the analyte bound to the plurality of detection spots, wherein a darkness of a detection spot of the plurality of detection spots is indicative of a concentration of the analyte.

19. The method of claim 18, further comprising determining the concentration of the analyte by measuring the initial rate of darkening of the plurality of detection spots.

20. The method of claim 18, further comprising determining the concentration of the analyte by subtracting nonspecific darkening of non-capture areas of the detection zone from the darkening of the plurality of detection spots.
